(19) United States
(12) Patent Application Publication
(10) Pub. No.: US 2010/0106161 A1
(43) Pub. Date: Apr. 29, 2010

(54) Surgical methods, devices, and kits

(76) Inventor: Marwan Tabbara, Miami, FL (US)

Correspondence Address: Cuenot, Forsythe & Kim, LLC, 3319 State Road 7, Suite 301, Wellington, FL, 33449, US

(21) Appl. No.: 12/604,988

(22) Filed: Oct. 23, 2009

Related U.S. Application Data
(60) Provisional application No. 61/107,808, filed on Oct. 23, 2008.

(51) Int. Cl. A61M 25/00

(52) U.S. Cl. 606/108

(57) ABSTRACT

Devices and techniques for establishing button-hole openings in arteriovenous fistula (or other target site located in the interior of a subject's body), without requiring the same nurse or technician to prepare the patient in the first several sessions, utilizing or encompassing a first button-hole forming device including in sequence: an external connection component; a scar-tissue forming component; and an indwelling cannula component. The first button-hole forming device has a lumen running through the longitudinal axis of the tube such that blood can be transported therethrough to or from the fistula (or other target site located in the interior of a subject's body) to a hemodialysis machine or other device. The devices and techniques also utilize or encompass a support device for supporting the first button-hole forming device at a constant angle relative to the plane formed by the subject's skin proximate to the fistula (or other target site located in the interior of a subject's body) when the first button-hole forming device is secured to the subject's body with the indwelling cannula component inserted into the fistula (or other target site located in the interior of a subject's body) such that a stable button-hole opening can be formed in a patient.

### CROSS-REFERENCE TO RELATED APPLICATIONS

The present application claims priority from U.S. provisional patent application Ser. No. 61/107,808 filed on Oct. 23, 2008, which is incorporated in its entirety herein by reference.

### FIELD OF THE INVENTION

The invention relates generally to the fields of medicine, kidney disease, hemodialysis, and vascular surgery. More particularly, the invention relates to methods and devices for forming a button-hole type opening in an arteriovenous (AV) fistula or other target site located in the interior of a subject's body.

### BACKGROUND

In renal failure patients, poor kidney function results in the accumulation of waste products and excess water in the blood. Hemodialysis uses an “artificial kidney” to remove these waste products and excess water from a patient's blood. In this procedure, the patient's blood is flowed over one side of a semi-permeable membrane while a dialysate is flowed in an opposite direction on the other side of the membrane. Osmotic pressure causes the waste products and excess water to selectively flow across the membrane into the dialysate to reduce their concentration in the patient's blood.

Over the last several decades numerous technological improvements have made this procedure safer and more effective. Nonetheless, because a large amount of blood must be transferred between a patient and a dialysis machine, it remains subject to complications such as infection and bleeding. It is also inconvenient to patients as it requires about 2-4 visits to a dialysis center each week.

To reduce complications and inconvenience, a number of methods and devices have been developed which facilitate repeated access to a patient's blood. These include intravenous catheters, synthetic grafts, and AV fistula (or other target site located in the interior of a subject's body)s. AV fistula (or other target site located in the interior of a subject's body) are generally preferred because they are longer-lasting and have fewer problems compared to intravenous catheters or synthetic grafts.

An AV fistula (or other target site located in the interior of a subject's body) is a large and strong distended vessel created surgically by connecting a vein and an artery, usually in the forearm or other site in the arm. Although it might take weeks or months to fully “mature,” once established, an AV fistula (or other target site located in the interior of a subject's body) provides a convenient, long-lasting site through which high volumes of blood can be removed and returned to a patient during hemodialysis. A preferred way to access blood through an AV fistula (or other target site located in the interior of a subject's body) is to create a “button hole” through its wall. Once properly formed, instead of a sharp needle, a blunt needle or cannula can be used for all future dialysis sessions. The button hole opening is particularly convenient because it does not require clotting to stop the bleeding once the needle or cannula is removed. Rather, within about 5 minutes from removal of the needle or cannula, the button hole collapses and seals itself (perhaps via a flap formed in the fistula (or other target site located in the interior of a subject's body)). The major advantages of using the “button-hole” method, as opposed to the traditional “create a new puncture every session” method, are that there is less trauma for the patient, less aneurysm formation, and a lower incidence of infection. Moreover, the button-hole method allows use of a blunt needle which makes the cannulation process much less painful compared to using a sharp needle. Notably, it also makes home dialysis possible without the help of a technician or nurse.

Despite these advantages, very few fistula (or other target site located in the interior of a subject's body) are accessed by the button-hole technique because proper formation requires exacting precision—that is, the fistula (or other target site located in the interior of a subject's body) has to be repeatedly punctured in precisely the same location and angle (about 25°) over several sessions. While this precision can often be achieved by the same technician or nurse, dedicating the same nurse or technician for 2 weeks is very difficult because of the way most dialysis centers are staffed.

### SUMMARY

The invention is based on the development of new devices and techniques for establishing button-hole openings in AV fistula (or other target site located in the interior of a subject's body) without requiring the same nurse or technician to prepare the patient in the first several sessions. The devices and techniques are also advantageous because they can be used for vascular access (e.g., for hemodialysis) while positioned in a patient for the period in which the button-hole opening is being formed.

In one aspect, the invention features a kit for forming a button hole-type scar-tissue tunnel track in a subject. The kit can include: at least a first button-hole forming device including in sequence: an external connection component; a scar-tissue forming component; and an indwelling cannula component. The first button-hole forming device can have a lumen running through the longitudinal axis of the tube such that blood can be transported therethrough to or from the fistula (or other target site located in the interior of a subject's body) to a hemodialysis machine or other device. The external connection component can include: (a) a first aperture at one end of the lumen, the first aperture being directly or indirectly connectable to a line in fluid communication with a hemodialysis machine and (b) a material defining a first portion of the lumen. The scar-tissue forming component can include a second portion of the lumen and an outer surface that can include a biocompatible material adapted to form a button hole-type scar tissue tunnel track in the subject. The indwelling cannula component can include a third section of the lumen and a second aperture at the end of the lumen opposite the first aperture, the indwelling cannula component can be adapted to facilitate positioning in, and in fluid communication, with an arterio-venous fistula (or other target site located in the interior of a subject's body) of the subject. The indwelling cannula component can have a width smaller than that of the scar-tissue forming component (e.g., when measured transverse to the longitudinal axis).

In variations of the foregoing, the outer surface of the scar-tissue forming component can have a width of between about 1.5 and 3.5 mm (e.g., between about 1.8 and 2.5 mm) and a length of between about 3.0 and 12.0 mm (e.g., between about 4.0 and 8.0 mm). The outer surface of the scar-tissue forming component can include a wrapping made of a biocompatible material such as a prolene suture, and/or an attached suture and needle for suturing the first button-hole forming device to the subject.

The external connection component can be at least partially transparent or translucent such that any blood within the portion of the lumen in this component can be detected visually. The material defining the first portion of the lumen can be adapted to allow reversible opening and closing of the first portion of the lumen (e.g., it can be flexible plastic that can be closed by tying with a suture or using a clamp). At least one of the components selected from the group consisting of the external connection component, the scar-tissue forming component, and the indwelling cannula component can include (e.g., be impregnated with) an anti-microbial agent (e.g., silver, nanoparticles, and polymers).

The first button-hole forming device can be cylindrical and/or have a length of between 7 and 14 centimeters, and/or the lumen can have a length of between 7 and 14 centimeters and/or a diameter of between 1 and 4 millimeters. The first button-hole forming device can be sterilized (e.g., by irradiation) and/or included within a hermetically sealed easy-open package. In one variation, the first button-hole forming device can be marked with a first identifier and the kit can further include a second button-hole forming device identical to the first button-hole forming device except being marked with a second identifier differing from the first identifier. The first and second identifiers can, e.g., include different colors such as red and blue.

The kit can further include a support device for supporting the first button-hole forming device at a constant angle (e.g., between 20 and 40 degrees, or between 25 and 35 degrees for a forearm fistula (or other target site located in the interior of a subject's body); 70-90 degrees for accessing the peritoneal space via the abdominal wall) relative to the plane formed by the subject's skin proximate to the fistula (or other target site located in the interior of a subject's body) when the first button-hole forming device is secured to the subject's body with the indwelling cannula component inserted into the fistula (or other target site located in the interior of a subject's body). The support device can be integrated with the button-hole forming device or included in the kit as a separate component. The support device can be cylindrical in shape and/or can include a component for engaging the first button-hole forming device. The component for engaging the first button-hole forming device can include a shape complementary to the shape of the portion of the first button-hole forming device that engages the component for engaging the first button-hole forming device. The support device can also include a suture and needle for securing the support device to the subject.

In another aspect, the invention features a method for forming a button hole-type scar-tissue tunnel track in a subject. The method can include the steps of: (a) obtaining a first button-hole forming device as described above and (b) securing the button-hole forming device to a patient with the indwelling cannula component at least partially inserted into a target site in the interior of the subject's body, the scar-tissue forming component at least partially positioned in the subcutaneous tissue located between the subject's skin and the target site, and the connection component positioned further to the outside of the subject's skin, wherein the button-hole forming device is held at a constant angle relative to the plane formed by the subject's skin proximate to the target site for between 10 and 20 days.

Unless otherwise defined, all technical terms used herein have the same meaning as commonly understood by one of ordinary skill in the art to which this invention belongs. Commonly understood definitions in surgery can be found in Steadman's Surgery Words, S. Kovacs (ed.), Williams & Wilkins, 1997.

Although methods and materials similar or equivalent to those described herein can be used in the practice or testing of the present invention, suitable methods and materials are described below. All publications mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

FIG. 1 is a schematic illustration of a button-hole preparation device.

FIG. 2 is a schematic illustration of two button-hole preparation devices inserted through the skin of a patient.

FIG. 3 is a schematic illustration of a button-hole preparation device inserted into an AV fistula (or other target site located in the interior of a subject's body) in a limb of a patient.

### DETAILED DESCRIPTION

The invention encompasses methods, devices, and kits for creating a button-hole opening in an AV fistula (or other target site located in the interior of a subject's body). The below described preferred embodiments illustrate adaptation of these methods, devices, and kits. Nonetheless, from the description of these embodiments, other aspects of the invention can be made and/or practiced based on the description provided below.

### General Methods

Methods involving conventional surgical techniques are described herein. Such techniques can be practiced by qualified surgeons and are also described in Way and Doherty, Current Surgical Diagnosis and Treatment, McGraw-Hill/Appleton & Lange, 11th edition (Sep. 24, 2002). Vascular surgical techniques are described in more detail in Rutherford's Textbook on Vascular Surgery, 5th ed., Robert B. Rutherford, WB Saunders Co., 2000.

### Kits and Devices

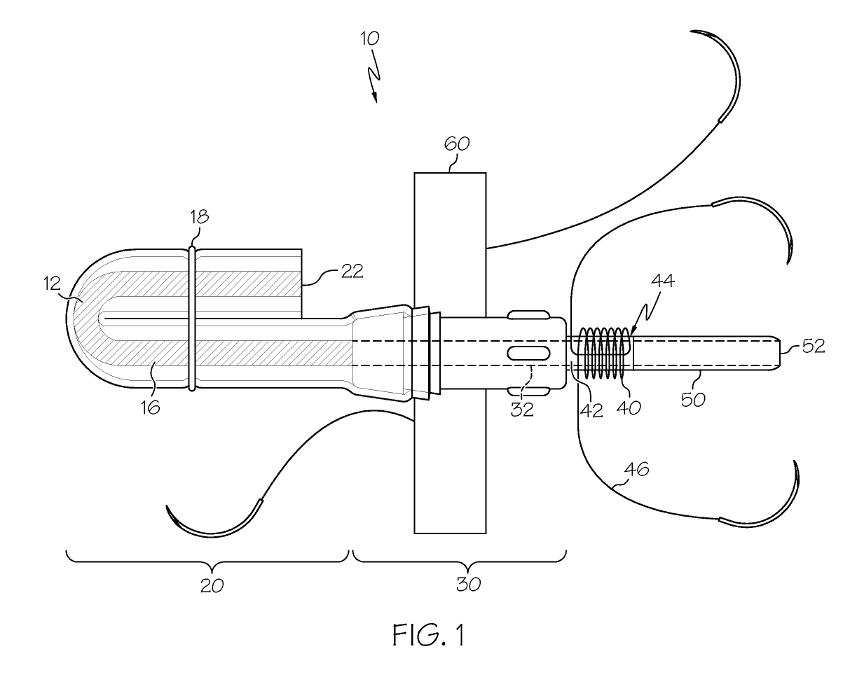

Referring now to FIG. 1, in an exemplary embodiment of the invention, a kit for forming a button hole-type scar-tissue tunnel track in a subject (e.g., a human being or a non-human animal such as an ape, monkey, cat, dog, pig, horse, cow, sheep, or goat) includes: at least a first button-hole forming device 10 that includes in sequence: an external connection component 20, a center component 30, a scar-tissue forming component 40, and an indwelling cannula component 50. The first button-hole forming device 10 has a length and defines a lumen 12 through the longitudinal axis of the tube 10.

The external connection component 20 includes: (a) a first aperture 22 at one end of the lumen 12. The first aperture 22 is directly or indirectly connectable to a line in fluid communication with a hemodialysis machine or blood analysis machine (such as a glucose monitor). The external connection component 20 can be made of any suitable material and can be of any dimension suitable for permitting blood flow from a fistula (or other target site located in the interior of a subject's body) to an external blood processing or testing machine such as a hemodialysis machine. Generally, it would take the form of plastic tubing having a length greater than 3 cm (e.g., greater than 3, 4, 5, 6, 7, 8, 9, 10 cm), and outer diameter of 1.2 to 3 times the diameter of the lumen 12. Shorter lengths which can still be reversibly closed are generally preferred for the convenience of the subject who would need to wear the tube 10 until formation of the button-hole opening. The first portion of the lumen 16 can be formed by a material such as plastic tubing that is flexible enough to be closed by tying off with a knotted suture, or using a clip or clamp.

The external connection component 20 can be at least partially transparent or translucent such that any blood within the portion of the lumen 22 within the external connection component 20 can be detected visually. The material defining the center component 30 and/or the first portion of the lumen 16 can be adapted to allow reversible opening and closing of the first portion of the lumen 16 (e.g., it can be flexible plastic that can be closed by tying with a suture or using a clamp). At least one of the components selected from the group consisting of the external connection component 20, the center component 30, the scar-tissue forming component 40, and the indwelling cannula component 50 can include (e.g., be impregnated with) an anti-microbial agent.

The center component 30 includes a second portion of the lumen 32. Although shown in the embodiments of FIGS. 1-3, it might be excluded in other embodiments. The center component 30 can be made of any suitable material and can be of any dimension suitable for permitting blood flow through second portion of the lumen 32. It can also be fashioned to facilitate securing the first button-hole forming device 10 to a support device 60. It can, for example, be made of a rigid plastic material and have a length of 1 to 4 cm (e.g., 2.5 cm). The center component 30 can have, measured transverse to the longitudinal axis of the tube 10, a width larger than the width of the scar-tissue forming component 40.

The scar-tissue forming component 40 can include a third portion of the lumen 42 and an outer surface 44 that can include a biocompatible material (e.g., prolene, silk, Dacron, nylon, and PTFE) adapted to form a button hole-type scar tissue tunnel in the subject. In certain embodiments, the outer surface 44 of the scar-tissue forming component 40 can have a width that supports the development of a stable and usable button-hole type opening, e.g., between about 1.5 and 3.5 mm (e.g., between about 1.8 and 2.5 mm; or 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, or 3.6 mm) and a length of between about 3.0 and 12.0 mm (e.g., between about 4.0 and 8.0 mm; or 2.5, 3, 4, 5, 6, 7, 8, 9, 10, 11, 12, or 12.5 mm). The device 10 can be made with scar-tissue forming component 40 of various lengths such that a surgeon can select the length that most closely approximates the distance between the wall of the fistula (or other target site located in the interior of a subject's body) and the top of the subcutaneous tissue. As shown in FIG. 1, the outer surface 44 can include a wrapping made of a biocompatible material such as a prolene suture, and/or an attached suture and needle 46 for suturing the first button-hole forming device to the subject.

The indwelling cannula component 50 includes a fourth section of the lumen and a second aperture 52 at the end of the lumen 12 opposite the first aperture 22. The indwelling cannula component 50 is configured such that it can be positioned in and in fluid communication with an arterio-venous fistula (or other target site located in the interior of a subject's body) of the subject, and, measured transverse to the longitudinal axis, can have a width smaller than that of the scar-tissue forming component 40. The indwelling cannula component 50 can be made of any suitable material and can be of any dimension suitable for permitting blood flow through a forth portion of the lumen and allowing access to the blood within the fistula (or other target site located in the interior of a subject's body). It can, for example, be made of a biocompatible material (e.g., plastic used to make intravenous angiocatheters) formed into a tubular shape.

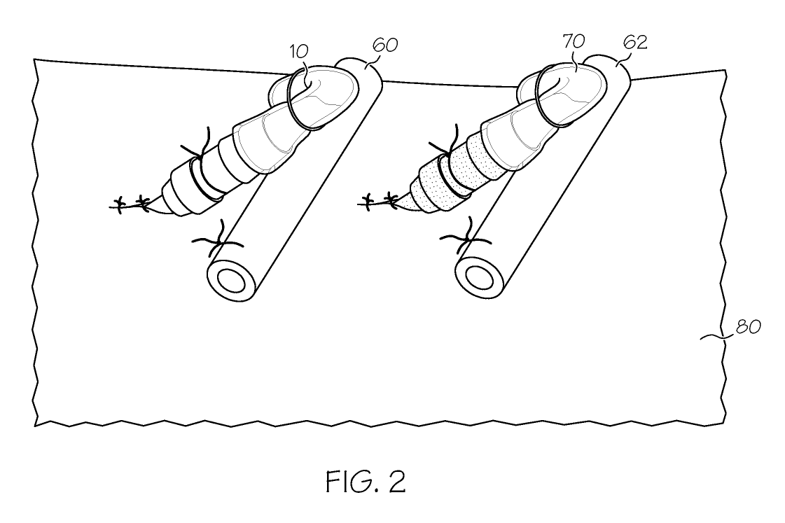

The first button-hole forming device 10 can be cylindrical and/or have a length of between 7 and 14 centimeters, and/or the lumen 12 can have a length of between 7 and 14 centimeters and/or a diameter of between 1 and 4 millimeters. The first button-hole forming device 10 can be sterilized (e.g., by irradiation) and/or included within a hermetically sealed easy-open package. In one variation, referring to FIG. 2, the first button-hole forming device 10 can be marked with a first identifier and the kit can further include a second button-hole forming device 70 identical to the first button-hole forming device except being marked with a second identifier (shown as stippling in FIG. 2) differing from the first identifier. The first and second identifiers can be different colors such as red and blue.

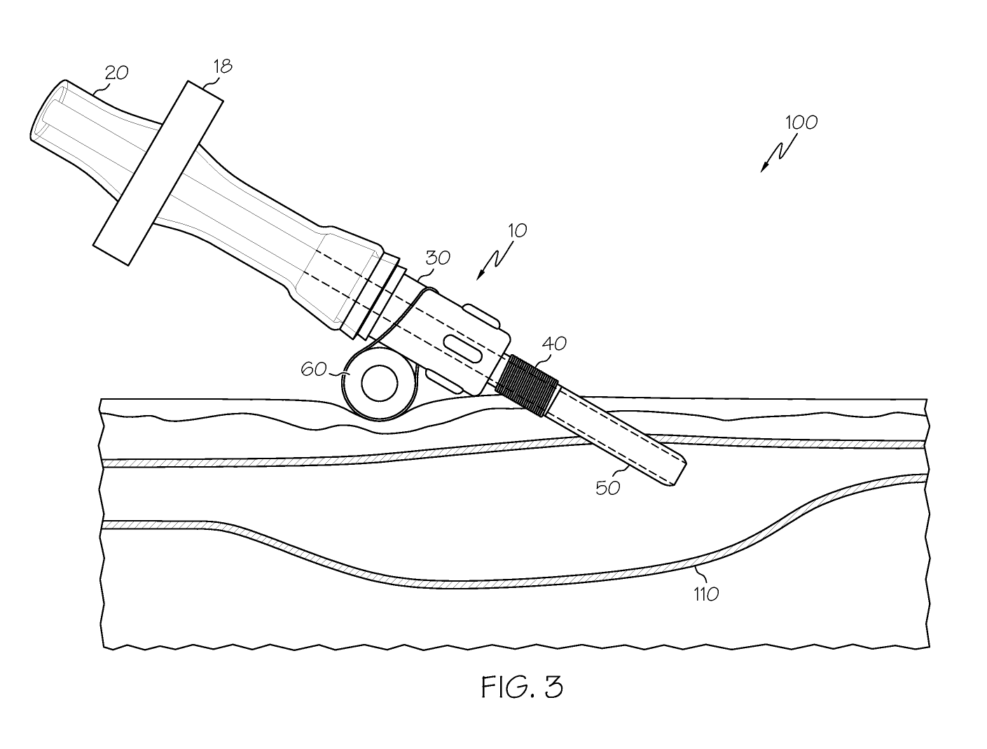

Referring to FIG. 3, the kit can further include a support device 60 for supporting the first button-hole forming device 10 at a constant angle (e.g., between 20 and 40 degrees; between 25 and 35 degrees; or 20, 21, 22, 23, 24, 25, 26, 27, 28, 29, 30, 31, 32, 33, 34, 35, 36, 37, 38, 39, or 40 degrees) relative to the plane formed by the subject's skin proximate to the fistula (or other target site located in the interior of a subject's body) 110 when the first button-hole forming device 10 is secured to the subject's body with the indwelling cannula component 50 inserted into the fistula (or other target site located in the interior of a subject's body) 110. The support device 60 can be integrated with the button-hole forming device 10 or included in the kit as a separate component. The support device 60 can be cylindrical in shape and/or can include a component for engaging the first button-hole forming device. The component for engaging the first button-hole forming device can include a shape complementary to the shape of the portion of the first button-hole forming device that engages the component for engaging the first button-hole forming device. The support device 60 can also include a suture and needle for securing the support device 60 to the subject.

### Methods of Forming a Button-Hole Type Opening

In another aspect, the invention features a method for forming a button hole-type scar-tissue tunnel track in a subject. The method can include the steps of: (a) obtaining a first button-hole forming device 10 as described above and (b) securing the button-hole forming device 10 to a patient with the indwelling cannula component 50 at least partially inserted to an arterio-venous fistula (or other target site located in the interior of a subject's body) 110 of the subject, with the scar-tissue forming component 40 at least partially positioned in the subcutaneous tissue located between the subject's skin and the fistula (or other target site located in the interior of a subject's body) 110, the center component 30 positioned outside and adjacent to the subject's skin, and the external connection component 20 positioned further to the outside of the subject's skin, wherein the button-hole forming device 10 is held at a constant angle relative to the plane formed by the subject's skin proximate to the fistula (or other target site located in the interior of a subject's body) until a stable button-hole type scar-tissue track develops, e.g., for between 10 and 20 days (for example, at least 10, 11, 12, 13, 14, 15, 16, 17, 18, 19, or 20 days)

### EXAMPLES

### Example 1

### Preparing a Button-Hole Opening in an AV Fistula (or Other Target Site Located in the Interior of a Subject's Body)

In an exemplary embodiment of the invention, a hemodialysis sheath is inserted into a patient's AV fistula (or other target site located in the interior of a subject's body). The sheath is a catheter such as can be a HEMO*CATH 7F® (Angiodynamics) that has been cropped to remove the peripheral part of the sheath leaving about a 3 cm length. Ten to twelve mm of the portion of the sheath immediately inferior to the hub is wound with polypropylene surgical monofilament (prolene wrapping). The prolene wrapping provides a higher friction surface which helps secure the device to the insertion site and also with the formation of the button-hole type scar-tissue tunnel track. The end of the prolene wrapping is used to suture the device to the subcutaneous tissue. The sheath with an introducer therein is advanced into a patient's fistula (or other target site located in the interior of a subject's body) at about a 25-40° (e.g., 24°, 25°, 30, 35°, 40°, or 41°) angle relative to the surface of the skin and in the direction of the blood flow (typically at 2 places on the fistula (or other target site located in the interior of a subject's body), approximately 5 cm apart). The introducer stiffens the soft sheath and provides a better profile at the end of the sheath for easier advancement. This is all done over a guide wire. The exposed end of the sheath tubing is then closed off, e.g., by bending the tube in half at about ¼″ from the end and tying it off with a black strong silk suture. Other types of closure devices such as a clip or clamp might also be used. The exposed end of tubing is then sutured to the patient's skin to secure the device. A spacer secured to the skin is placed under the exposed end of the tubing to maintain the angle. A plastic cylinder about 5 mm (e.g., 3-7 mm) in diameter and about 2-3 cm in length can be used as the spacer. Sheaths are left subcutaneously in the patient for about 2 weeks, after which the sheath is removed leaving a formed button-hole which can then be repeatedly accessed using a blunt needle, cannula, or like device. Thus, the need for repeated punctures which can vary in location and angle are avoided.

### Example 2

### Kit for Making a Button-Hole Opening in a Fistula (or Other Target Site Located in the Interior of a Subject's Body) for Hemodialysis

A kit for making a button-hole opening in a fistula (or other target site located in the interior of a subject's body) for hemodialysis can include a button-hole preparation device described in (b) below and, optionally one or more (e.g., 1, 2, 3, or 4) of the other components listed below:

(a) two buttresses suitable for holding a button-hole preparation device at a 25-40° angle (relative to patients skin surface) and being sutured to the skin (e.g., a 1-2 cm long plastic cylinder about 0.3-0.6 cm in diameter).

(b) two button-hole preparation devices which are made of two hemodialysis sheaths (e.g., one red and one blue) each wrapped near the hub with a polypropylene suture with attached needles (e.g., 3-0 Prolene SH non-cutting [round] needle). The devices have a means for closing the open end of the sheaths that would be outside the patient when inserted in a fistula (or other target site located in the interior of a subject's body). The closure means could take the form of a 2-0 silk tie wrapped around the folded end of the sheaths.

(c) a dilator (e.g., 7F) to dilate the track and fistula (or other target site located in the interior of a subject's body) prior to inserting the button-hole preparation devices into the fistula (or other target site located in the interior of a subject's body).

(d) a needle for cutting a skin tunnel and entering the fistula (or other target site located in the interior of a subject's body) over 0.018 wire, and making a flap in the fistula (or other target site located in the interior of a subject's body) (e.g., a needle that comes with a 16 gauge angiocatheter).

(e) a micropuncture kit to assist in performing a fistulogram prior to making the button holes, and to pass a J wire (e.g., 0.35) and slide a button-hole preparation device over it.

### Example 3

### Making a Button-Hole Preparation Device

Marks are made on a hemodialysis sheath 1 cm and 3 cm below the hub. A 3-0 prolene suture with a tapered SH needle is passed into the sheath 14 mm from the hub and wrapped around (i) the free end of the suture (which is positioned parallel to the length of the sheath) and (ii) the area of the sheath just below the hub as shown. The dilator is passed through the hub and secured to the sheath. Holding the free end with index finger and thumb, the end with the needle is wrapped around the sheath several times so that about 10-12 mm of the sheath just below the hub is covered with polypropylene monofilament. The free end and needle end are then tied together to secure the prolene wrapping. The sheath is then cut flushly at about 3 cm below the hub (at the second mark). The total length of the remaining part of the sheath below the hub is about 3 cm, of which about 2.5 cm will be inside the fistula (or other target site located in the interior of a subject's body) when inserted.

### Example 4

### Procedure for Making a Button-Hole in a Fistula (or Other Target Site Located in the Interior of a Subject's Body)

Ultrasound imaging is used to assess the fistula (or other target site located in the interior of a subject's body). The fistula (or other target site located in the interior of a subject's body) should be at least 8 mm in diameter. If it is not, balloon angioplasty maturation (BAM) should first be performed to increase the size of the fistula (or other target site located in the interior of a subject's body). The fistula (or other target site located in the interior of a subject's body) is accessed as peripheral as possible. A fistulogram is performed and pressure is determined. If there are any problems with the fistula (or other target site located in the interior of a subject's body) or the pressure is above 50 mmHg, the procedure is not performed until these problems are resolved (e.g., with angioplasty/stenting).

Two spots are marked with a line perpendicular to the fistula (or other target site located in the interior of a subject's body) and a dot placed over the needle entry site. The area is infiltrated with 1% xylocalne (no epinephrine). No incision is made. With ultrasound guidance, the fistula (or other target site located in the interior of a subject's body) is cannulated with a 21 gauge micropuncture needle 25-40 degrees exactly in the middle interiorly, traveling 5 mm in a subcutaneous tunnel. A 0.018 guide wire is advanced and 16 gauge needle passed with the bevel down. After going through the vein (the outflow of the fistula (or other target site located in the interior of a subject's body)), the 16 gauge needle is removed and a 4F coaxial exchange dilator is placed over the wire. An incision at least 5 mm is made in the skin with a number 11 blade. The 0.18 wire and inner dilator are removed, and the fistula (or other target site located in the interior of a subject's body) is assessed by taking pressure and performing a fistulogram. A 0.35 J wire with the 7F dilator is advanced followed by the already wrapped and cut sheath (a button-hole preparation device described in Example 3).

The button-hole preparation device is placed in the hole with the prolene wrapping fully in the subcutaneous space. The sheath is pushed in a twisting movement and advanced to the hub. With a 7F sheath, the skin is cut at least 5 mm in-order to avoid the sheath holding at that level. The needle connected to the 3-0 prolene is passed in the tissue around the sheath and tied going centrally.

The 5F inner dilator is kept and the J wire removed. The inner dilator is pulled back so only few mm protrudes from the sheath, and blood return from the inner dilator is checked. A contrast agent is injected to make sure there is no extravasation. The 2-0 tie is used to make a snug tie over the plastic part of the sheath. The plastic part of the sheath is then folded over and secured with a tie. Any excess plastic part can be cut. Using ultrasound guidance, the sheath is aligned in the middle of the fistula (or other target site located in the interior of a subject's body). The skin under the sheath is then marked to assure that the side to side angle is correct, and the area is infiltrated with a local anesthetic. The needle of a 3-0 prolene suture is then passed through a plastic buttress spacer subcutaneously and back in the plastic buttress then around the sheath hub and tied to maintain a precise angle (preferably between 20-30°) of the cannulation needle approaching the button hole in the future.

### Example 5

### Button-Hole Opening for Diabetic

A single button-hole opening is made in a fistula (or other target site located in the interior of a subject's body) as described above. Blood glucose can be monitored and insulin injected through the button-hole opening (e.g., manually or with an automatic device).

### Example 6

### Button-Hole Opening for Access to the Peritoneum

A single button-hole opening is made in the abdominal wall between the skin and peritoneal cavity using the methods described above. Fluids and drugs (e.g., insulin) can be delivered to the peritoneum via the button-hole forming device while the device is in place to form the button-hole opening. After formation of the button-hole opening, a blunt needle can be pushed through the tract to deliver drugs or any fluid into the peritoneal cavity.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A kit for forming a button hole-type scar-tissue tunnel track in a subject, the kit comprising:
at least a first button-hole forming device comprising in sequence: an external connection component, a scar-tissue forming component, and an indwelling cannula component,
the first button-hole forming device having a length and defining a lumen through the longitudinal axis of the tube;
the external connection component comprising: (a) a first aperture at one end of the lumen, the first aperture being directly or indirectly connectable to a line in fluid communication with a hemodialysis machine and (b) a material defining a first portion of the lumen;
the scar-tissue forming component comprising a second portion of the lumen and an outer surface comprising a biocompatible material adapted to form a button hole-type scar tissue tunnel in the subject; and
the indwelling cannula component comprising a third section of the lumen and a second aperture at the end of the lumen opposite the first aperture, the indwelling cannula component adapted to being positioned in and in fluid communication with an arterio-venous fistula (or other target site located in the interior of a subject's body) of the subject.

2. The kit of claim 1, wherein the outer surface of the scar-tissue forming component has a width between about 1.5 and 3.5 mm and a length of between about 3.0 and 12.0 mm.

3. The kit of claim 1, wherein the width of the outer surface of the scar-tissue forming component is between about 1.8 and 2.5 mm and a length of between about 4.0 and 8.0 mm.

4. The kit of claim 1, wherein the scar-tissue forming component comprises a wrapping made of a biocompatible material.

5. The kit of claim 4, wherein the wrapping comprises a prolene suture.

6. The kit of claim 1, the scar-tissue forming component comprises an attached a suture and needle for suturing the first button-hole forming device to the subject.

7. The kit of claim 1, wherein the external connection component is at least partially transparent or translucent such that any blood within the portion of the lumen within the external connection component can be detected visually.

8. The kit of claim 1, wherein the material defining the first portion of the lumen is adapted to allow reversible opening and closing of the first portion of the lumen.

9. The kit of claim 1, wherein at least one of the components selected from the group consisting of the external connection component, the scar-tissue forming component, and the indwelling cannula component comprises an anti-microbial agent.

10. The kit of claim 1, wherein the first button-hole forming device is cylindrical and has a length of between 7 and 14 centimeters and the lumen has a length of between 7 and 14 centimeters and a diameter of between 1 and 4 millimeters.

11. The kit of claim 1, wherein the first button-hole forming device has been sterilized.

12. The kit of claim 11, wherein the first button-hole forming device is comprised within a hermetically sealed easy open package.

13. The kit of claim 1, wherein the first button-hole forming device is marked with a first identifier and the kit further comprises a second button-hole forming device identical to the first button-hole forming device except being marked with a second identifier differing from the first identifier.

14. The kit of claim 13, wherein the first and second identifiers comprise different colors.

15. The kit of claim 1, wherein the kit further comprises a support device for supporting the first button-hole forming device at a constant angle relative to the plane formed by the subject's skin proximate to the fistula (or other target site located in the interior of a subject's body) when the first button-hole forming device is secured to the subject's body with the indwelling cannula component inserted into the fistula (or other target site located in the interior of a subject's body).

16. The kit of claim 15, wherein the angle is between 20 and 40 degrees.

17. The kit of claim 15, wherein the angle is between 25 and 35 degrees.

18. The kit of claim 15, wherein support device comprises a cylindrical shape.

19. The kit of claim 15, wherein support device comprises a component for engaging the first button-hole forming device, the component for engaging the first button-hole forming device comprising a shape complementary to the shape of the portion of the first button-hole forming device that engages the component for engaging the first button-hole forming device.

20. The kit of claim 15, wherein the support device comprises a suture and needle for securing the support device to the subject.

21. A kit for forming a button hole-type scar-tissue tunnel track in a subject, the kit comprising:
at least a first button-hole forming device comprising in sequence: an external connection component, a scar-tissue forming component, and an indwelling cannula component,
the first button-hole forming device having a length and defining a lumen through the longitudinal axis of the tube;
the external connection component comprising: (a) a first aperture at one end of the lumen, the first aperture being directly or indirectly connectable to a line in fluid communication with a hemodialysis machine and (b) a material defining a first portion of the lumen;
the scar-tissue forming component comprising a second portion of the lumen and an outer surface comprising a biocompatible material adapted to form a button hole-type scar tissue tunnel in the subject; and
the indwelling cannula component comprising a third section of the lumen and a second aperture at the end of the lumen opposite the first aperture, the indwelling cannula component adapted to being positioned in and in fluid communication with an arterio-venous fistula (or other target site located in the interior of a subject's body) of the subject, and, measured transverse to the longitudinal axis, having a width smaller than that of the scar-tissue forming component,
wherein the width of the outer surface of the scar-tissue forming component is between about 1.8 and 2.5 mm and a length of between about 4.0 and 8.0 mm; the external connection component is at least partially transparent or translucent such that any blood within the portion of the lumen within the external connection component can be detected visually; the material defining a first portion of the lumen is adapted to be allow reversible opening and closing of the first portion of the lumen; the first button-hole forming device is sterilized, comprised within a hermetically sealed easy open package, and cylindrical with a length of between 7 and 14 centimeters; and the lumen has a length of between 7 and 14 centimeters and a diameter of between 1 and 4 millimeters.

22. A method for forming a button hole-type scar-tissue tunnel track in a subject, the method comprising the steps of:
(a) obtaining a first button-hole forming device comprising in sequence: a connection component, a scar-tissue forming component, and an indwelling cannula component,
the first button-hole forming device having a length and defining a lumen through the longitudinal axis of the tube;
the connection component comprising: a first aperture at one end of the lumen, and a material defining a first portion of the lumen;
the scar-tissue forming component comprising a second portion of the lumen and an outer surface comprising a biocompatible material adapted to form a button hole-type scar tissue tunnel in the subject; and
the indwelling cannula component comprising a third section of the lumen and a second aperture at the end of the lumen opposite the first aperture, and
(b) securing the button-hole forming device to a patient with the indwelling cannula component at least partially inserted into a target site in the interior of the subject's body, the scar-tissue forming component at least partially positioned in the subcutaneous tissue located between the subject's skin and the target site, and the connection component positioned further to the outside of the subject's skin, wherein the button-hole forming device is held at a constant angle relative to the plane formed by the subject's skin proximate to the target site for between 10 and 20 days.
